(19) United States
(12) Patent Application Publication
(10) Pub. No.: US 2014/0120143 A1
(43) Pub. Date: May 1, 2014

(54) KIT FOR PERSONALIZING THE COLOR OF A TOPICAL SKIN COMPOSITION

(71) Applicant: MARY KAY INC., Dallas, TX (US)

(72) Inventors: Mauricio Castro, Carrolton, TX (US); Amit Patel, Dallas, TX (US)

(73) Assignee: MARY KAY INC., Dallas, TX (US)

(21) Appl. No.: 14/063,798

(22) Filed: Oct. 25, 2013

Related U.S. Application Data
(60) Provisional application No. 61/718,915, filed on Oct. 26, 2012.

(51) Int. Cl. A61K 8/73; A61K 8/27; A61Q 1/02; A61K 8/29

(52) U.S. Cl. CPC A61K 8/731; A61K 8/29; A61K 8/27; A61Q 1/02
USPC 424/401; 424/63

(57) ABSTRACT

Disclosed is a kit for preparing a colored topical skin composition, the kit comprising a first composition comprising an aqueous continuous phase comprising at least 50% by weight of water and a discontinuous phase comprising titanium dioxide or zinc oxide or a combination thereof in an amount sufficient to impart a white color appearance to the first composition, wherein the titanium dioxide or zinc oxide is suspended within the aqueous continuous phase, and wherein the color of the first composition is white and a second composition in the form of a water-dissolvable film, wherein the film has a color other than white, wherein the second composition is capable of dissolving within the first composition and imparting a color other than white to the first composition when the first and second compositions are mixed together.

### CROSS REFERENCE TO RELATED APPLICATIONS

This application claims the benefit of U.S. Provisional Application No. 61/718,915 filed Oct. 26, 2012. The contents of the referenced application is incorporated into the present application by reference.

### BACKGROUND OF THE INVENTION

A. Field of the Invention

The present invention relates generally to a kit that can be used to personalize the color of a topical skin composition. The kit includes a first composition comprising an aqueous continuous phase comprising at least 50% by weight of water and a discontinuous phase comprising titanium dioxide or zinc oxide or a combination thereof in an amount sufficient to impart a white color appearance to the first composition, wherein the titanium dioxide or zinc oxide is suspended within the aqueous continuous phase, and wherein the color of the first composition is white, and a second composition in the form of a water-dissolvable film, wherein the film has a color other than white, wherein the second composition is capable of dissolving within the first composition and imparting a color other than white to the first composition when the first and second compositions are mixed together.

B. Description of Related Art

Topical skin formulations come in a wide variety of colors. In several instances, the same formulation is offered in numerous color shades in hopes of maximizing sales to end-users who often times have varying preferences to a particular color. For instance, beauty balm creams (“BB Creams”) that are currently offered can have upwards of 20 different color shades even though the underlying formulation is the same. Despite the wide color selection, an end user's particular skin condition (e.g., melisma, age spots, sun spots, dark spots, scars, erythemic skin, freckles etc.) or skin tone (light skin, medium skin, dark skin, uneven skin, etc.) may dictate the use of a particular color shade that is not offered for sale to disguise or reduce the appearance of a particular skin condition or to match a particular skin tone. This can lead to unsatisfied customers. Further, end-users may want to vary the color applied to their skin on a day to day basis and, as such, may have to purchase a multitude of different shades of the same composition to achieve a desired look for a given day, which can be expensive to the end user.

### SUMMARY OF THE INVENTION

The inventors have discovered a solution to the problems associated the number of shades of colors that are currently offered for a given product formulation. This solution resides in the combination of a particular skin formulation that has a white color and the use of colored water-dissolvable strips that can be dissolved into the skin formulation, thereby changing the skin formulation color from white to a desired color of the end-user. This allows for an end-user to purchase a single skin formulation and change the color of said formulation as desired by using the color strips.

In one aspect of the present invention, there is disclosed a kit for preparing a colored topical skin composition, the kit comprising a first composition comprising an aqueous continuous phase comprising at least 50% by weight of water and a discontinuous phase comprising titanium dioxide or zinc oxide or a combination thereof in an amount sufficient to impart a white color appearance to the first composition, wherein the titanium dioxide or zinc oxide is suspended within the aqueous continuous phase, and wherein the color of the first composition is white and a second composition in the form of a water-dissolvable film, wherein the film has a color other than white, wherein the second composition is capable of dissolving within the first composition and imparting a color other than white to the first composition when the first and second compositions are mixed together. The first composition can be formulated as a dispersion, emulsion, or gel. The amount of water in the first composition can include 55, 60, 65, 70, 75, 80, 85, and up 90% by weight, provided that there is a sufficient amount of titanium dioxide or zinc oxide or both within the composition that imparts a white color to said composition. In particular aspects, the amount of water can range from 60 to 75% by weight. With respect to the amount of titanium dioxide or zinc oxide or both to use, a sufficient amount is an amount that leads to the first composition having the appearance of a white color. “White” in this context generally means not only achromatic white but also whitish chromatic colors such as off-white, ivory, and light-gray. In particular aspects, the amount of titanium dioxide or zinc oxide can range from 5 to 25% by weight of the composition (w/w), or at least 5, 10, 15, 20, or 25% by weight of the composition, or 5 to 15% by weight or 8 to 12% by weight or around 10% by weight, although other amounts and ranges are contemplated. It was discovered by a ratio of around 5:1 to 15:1 of water to titanium dioxide or zinc oxide or both in a sufficient amount to impart a white color to the first composition. In particular aspects, the ratio can be around 5:1 to 10:1, or 6:1 to 7:1, or about 6.5:1. In certain aspects, the first composition can also include a gelling agent to aid in an efficient dispersion of the titanium dioxide or zinc oxide particles within the aqueous phase of the first composition. The amount of gelling agent can range as needed to create a suspension or dispersion and to create a desired tactile property and viscosity. In certain aspects, the amount can be from 0.1 to 10%, or 0.5 to 5%, or 0.5 to 2%. Given that the first composition can be a topical skin product, it can have additional attributes that may be desired by an end user. Such attributes can include moisturization, skin exfoliation, anti-wrinkle, anti-aging, revitalization, skin renewal attributes and the like. Therefore, the composition can be loaded with active ingredients to achieve a desired result (e.g., moisturizers, anti-oxidants, anti-aging agents, vitamins, minerals, essential oils, botanical extracts, chemical sunscreens, physical sunscreens (in some instances, the titanium dioxide or zinc oxide can provide both a white color and sunscreen properties to the composition), and the like). Further, the compositions can include thickening agents, silicone containing compounds, emulsifiers, preservatives, cosmetic ingredients, pharmaceutical ingredients, and the like. In one instance, for example, the composition can include 5 to 15% by weight of a moisturizer or a combination of moisturizers. In some instances, the water-dissolvable film can be a cellulose-based film. The cellulose-based film can be a hydroxypropyl cellulose (HPC) based film, a hydroxypropylmethyl cellulose (HPMC) based film, or a carboyxymethyl cellulose (CMC) based film or a combination thereof. In one instance, the water-dissolvable film comprises a pigment or a blend of pigments that are capable of imparting a color other than white to the film. Examples of colors for the film include red, green, blue, yellow, brown, black, pink, or any other desired color for the end-user. In this sense, the kit can have a multitude of different colored water-dissolvable films (e.g., in the form of small square, rectangular, triangular, or round cut-outs) that the user can combine within the formulation to create any desired color or shade of color. Further, the kit can include instructions that provide information on the amount of a film to use and the combination of various films that lead to a given color (e.g., the combination of yellow and blue forming a green composition). In some aspects, the water-dissolvable film has a thickness ranging from 1 to 3 millimeters, or from 1.5 to 2.5 millimeters, or approximately 2 millimeters. In some aspects, the first composition can be included in a first container or compartment and the second composition can be included in a second container or compartment. Further, a third container can be used to hold the first and second containers or to hold the first and second compartments. Additionally, the second container can include multiple second compositions of the same or varying colors. Alternatively, additional containers or compartments can be included to house additional second compositions of the present invention.

In another aspect of the present invention, there is disclosed a method of preparing or applying a composition to skin with the kits of the present invention. The method can include mixing a first composition of the present invention with a second composition. The second composition of the present invention can dissolve within the first composition of the present invention. This can change the color of the first composition from white to a second color desired or chosen by the end-user. In some instances, the color of the first composition, after mixing with the second composition, can be substantially identical to the color of a user's skin color. In other instances, the first composition, after mixing with the second composition, can be any variety of color, shade of color and the like (e.g., blue, green, red, yellow, brown, black, orange, light blue, dark blue, light green, dark green, light red, dark red, light yellow, dark yellow, light brown, dark brown, light orange, dark orange, etc.).

It is contemplated that any embodiment discussed in this specification can be implemented with respect to any method or composition of the invention, and vice versa. Furthermore, compositions of the invention can be used to achieve methods of the invention.

“Topical application” means to apply or spread a composition onto the surface of lips or skin, or keratinous tissue. “Topical skin composition” includes compositions suitable for topical application on lips or keratinous tissue. Such compositions are typically dermatologically-acceptable in that they do not have undue toxicity, incompatibility, instability, allergic response, and the like, when applied to lips or skin. Topical skin care compositions of the present invention can have a selected viscosity to avoid significant dripping or pooling after application to skin. “Keratinous tissue” includes keratin-containing layers disposed as the outermost protective covering of mammals and includes, but is not limited to, lips, skin, hair and nails.

The term “about” or “approximately” are defined as being close to as understood by one of ordinary skill in the art, and in one non-limiting embodiment the terms are defined to be within 10%, preferably within 5%, more preferably within 1%, and most preferably within 0.5%.

The term “substantially” and its variations are defined as being largely but not necessarily wholly what is specified as understood by one of ordinary skill in the art, and in one non-limiting embodiment substantially refers to ranges within 10%, within 5%, within 1%, or within 0.5%.

The terms “inhibiting” or “reducing,” or any variation of these terms, includes any measurable decrease or complete inhibition to achieve a desired result. The terms “promote” or “increase” or any variation of these terms includes any measurable increase or production of a protein or molecule (e.g., matrix proteins such as fibronectin, laminin, collagen, or elastin or molecules such as hyaluronic acid) to achieve a desired result.

The term “effective,” as that term is used in the specification and/or claims, means adequate to accomplish a desired, expected, or intended result.

The use of the word “a” or “an” when used in conjunction with the term “comprising” in the claims and/or the specification may mean “one,” but it is also consistent with the meaning of “one or more,” “at least one,” and “one or more than one.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The kits, compositions, and methods for their use can “comprise,” “consist essentially of,” or “consist of” and of the ingredients or steps disclosed throughout the specification. With respect to the transitional phase “consisting essentially of,” in one non-limiting aspect, a basic and novel characteristic of the kits, compositions, and methods disclosed in this specification includes the first composition having a white color and the second composition's ability to dissolve within the first composition and to change the color of the first composition from white to another desired or selected color.

Other objects, features and advantages of the present invention will become apparent from the following detailed description. It should be understood, however, that the detailed description and the examples, while indicating specific embodiments of the invention, are given by way of illustration only. Additionally, it is contemplated that changes and modifications within the spirit and scope of the invention will become apparent to those skilled in the art from this detailed description.

### DESCRIPTION OF ILLUSTRATIVE EMBODIMENTS

As noted above, one of the unique aspects of the present invention is an end-user's ability to create multiple colors and shades of colors of a topical skin composition from a single composition that has a white color. The ability to do this is driven by two separate compositions (a first and second composition), when combined, result in a change of color of the composition. Further, the color change is an even color change in that there is minimal coalescing of the colorants in the second composition, thereby reducing the appearance of color streaks and uneven color appearance in the resulting mixed colored composition. The following subsections describe non-limiting aspects of the present invention in further detail.

### A. First Composition

Without wishing to be bound by theory, three features of the first composition that allows for the invention to work are the structure of the composition, the amount of water in the composition, and the amount of titanium or zinc oxide in the composition. The composition is structured such that the external or continuous phase is water (e.g., an oil-in-water emulsion, an aqueous dispersion, or a gel). This along with the amount of water in the first composition allows for the water-dissolvable film or strip to efficiently dissolve within the first composition. It was discovered that the amount of water in the first composition should be at least 50% by weight of the composition. However, the upper limit level of water is capped by the presence of titanium dioxide or zinc oxide present within the first composition. In particular, a ratio of around 5:1 to 15:1 of water to titanium dioxide or zinc oxide or both allows for a sufficient amount of water to dissolve the water-dissolvable strip or film while still imparting a white color to the first composition. The third feature, as alluded to in the previous sentence, is the presence of titanium dioxide or zinc oxide in the first composition in an amount that results in the first composition having a white color. Further, the titanium dioxide or zinc oxide is suspended within the first composition. The use of suspended titanium dioxide or zinc oxide allows for the colors within the water-dissolvable strip or film to efficiently disperse within the first composition, after said strip or film has dissolved within the first composition. The use titanium dioxide or zinc oxide reduces the likelihood of chemical reactions with the colors from the water-dissolvable strip or film. Stated another way, the suspended titanium dioxide or zinc oxide provides a stable and low reactive white canvas for the colored water-dissolvable strips to change the color of the white canvas to a color desired by the end user.

In addition to the above features, the first composition can have additional ingredients. Such additional ingredients include gelling agents, viscosity modifying agents, active ingredients, additional sunscreens, and the like. With the inclusion of these additional ingredients, one can create an all-in-one composition for the end user, and the end user can then chance the color of the composition to a desired color to serve a particular purpose (e.g., to disguise blemishes or uneven skin tone, to add color to skin, etc.).

### B. Second Composition

The second composition of the present invention is a water-dissolvable strip or film that is capable of dissolving within the first composition. In addition to the water-dissolvability of the strip or film, a key feature of the second composition is its color. In this sense, it is contemplated that the second composition can have a variety of different colors, which when mixed with the first composition, results in a color change of the first composition from white to a desired color. The second composition can be easily colored with the use of colorants. There are a wide range of colorants that are commercially available that can be used to color the second composition (see International Cosmetic Ingredient Dictionary and Handbook (“CTFA”), 12^{th} Edition, Volume 3, pages 3174-3176 (2008), which is incorporated by reference). A person of skill in the art can easily mix and match such colorants to produce a particular color for the second composition of the present invention.

There are a variety of water-dissolvable films that are commercially available and that can be used with the first composition of the present invention. Examples of such commercially available films having various colors include those offered by Aicello (Japan) under the trade name Solublon® Water Soluble Film and MonoSol (USA) under the trade name MonoDose™. Further, the following publications provide explanations on the various processes that can be used to prepare water-dissolvable films, both of which are incorporated by reference: “Film-Forming Polymers in Fast-Dissolve Oral Films,” by Chen et al., a Dow Chemical Company publication (2006) (accessible at http://msdssearch.dow.com/PublishedLiteratureDOWCOM/dh_{—}0062/0901b803800629f0.pdf?fil epath=dowexcipients/pdfs/noreg/198-02161.pdf&fromPage=GetDoc) and “Edible Films Applications,” Methocel Food Gums (accessible at http://msdssearch.dow.com/PublishedLiteratureDOWCOM/dh_{—}0032/0901b80380032eff.pdf?file path=/194-01283.pdf&fromPage=GetDoc), both of which are incorporated by reference. Colorants can be added to the films by mixing said colorants into the aqueous film formulations prior to drying said films (see Legendre et al., “Formulation, Characterization, and Efficacy of an Adenosine-Container Dissolvable Film for a Localized Anti-Wrinkle Effect,” J. Cosmet Sci., 2007 March-April; 58(2):147-55, which is incorporated by reference). A further way to prepare a water-dissolvable strip or film of the present invention is provided in Example 3, which is derived from U.S. Publication 2009/0022700, which is incorporated by reference).

### C. Methods of Using the First and Second Compositions

Methods of using the first and second compositions are relatively straight forward. For instance, one can take a second composition film or strip having a given color and add it to the first composition and mix until the second composition is dissolved within the first composition (mixing can be done in the palm of one's hand or in a container and visual inspection can be used to determine when the second composition is fully dissolved). After mixing, if a desired color is obtained, then the user can apply the composition to skin. If, however, the desired color is not obtained, then the user can added additional films or strips (of the same color or of different colors from the first film or strip) and mix until the desired color is obtained.

### D. Kits

Kits are also contemplated as being used in certain aspects of the present invention. For instance, the first and second compositions of the present invention can be included in a single package or container. The package or container can include multiple compartments such as one for the first composition and one for the second composition. The kit can also include multiple second compositions having different colors, which would allow the user to create a wide array of colors and shades of colors when combined with the first composition. Also, the kit can include instructions, which could instruct a user on the amount of the first composition, the amount of the second composition, and the color of the second composition to add to the first composition to achieve a desired color or shade of color of the mixed/resulting colored composition. Further, the first composition can be included in a bottle, a metal tube, a laminate tube, a plastic tube, a dispenser, or a pressurized container. The second composition can be included in a compartment that can hold multiple strips or films of the same color or of varying colors.

### E. Amounts of Ingredients

It is contemplated that the first and second compositions of the present invention can include any amount of the ingredients discussed in this specification. The compositions can also include any number of combinations of additional ingredients described throughout this specification (e.g., pigments, or additional cosmetic or pharmaceutical ingredients). The concentrations of the any ingredient within the compositions can vary. In non-limiting embodiments, for example, the compositions can comprise, consisting essentially of, or consist of, in their final form, for example, at least about 0.0001%, 0.0002%, 0.0003%, 0.0004%, 0.0005%, 0.0006%, 0.0007%, 0.0008%, 0.0009%, 0.0010%, 0.0011%, 0.0012%, 0.0013%, 0.0014%, 0.0015%, 0.0016%, 0.0017%, 0.0018%, 0.0019%, 0.0020%, 0.0021%, 0.0022%, 0.0023%, 0.0024%, 0.0025%, 0.0026%, 0.0027%, 0.0028%, 0.0029%, 0.0030%, 0.0031%, 0.0032%, 0.0033%, 0.0034%, 0.0035%, 0.0036%, 0.0037%, 0.0038%, 0.0039%, 0.0040%, 0.0041%, 0.0042%, 0.0043%, 0.0044%, 0.0045%, 0.0046%, 0.0047%, 0.0048%, 0.0049%, 0.0050%, 0.0051%, 0.0052%, 0.0053%, 0.0054%, 0.0055%, 0.0056%, 0.0057%, 0.0058%, 0.0059%, 0.0060%, 0.0061%, 0.0062%, 0.0063%, 0.0064%, 0.0065%, 0.0066%, 0.0067%, 0.0068%, 0.0069%, 0.0070%, 0.0071%, 0.0072%, 0.0073%, 0.0074%, 0.0075%, 0.0076%, 0.0077%, 0.0078%, 0.0079%, 0.0080%, 0.0081%, 0.0082%, 0.0083%, 0.0084%, 0.0085%, 0.0086%, 0.0087%, 0.0088%, 0.0089%, 0.0090%, 0.0091%, 0.0092%, 0.0093%, 0.0094%, 0.0095%, 0.0096%, 0.0097%, 0.0098%, 0.0099%, 0.0100%, 0.0200%, 0.0250%, 0.0275%, 0.0300%, 0.0325%, 0.0350%, 0.0375%, 0.0400%, 0.0425%, 0.0450%, 0.0475%, 0.0500%, 0.0525%, 0.0550%, 0.0575%, 0.0600%, 0.0625%, 0.0650%, 0.0675%, 0.0700%, 0.0725%, 0.0750%, 0.0775%, 0.0800%, 0.0825%, 0.0850%, 0.0875%, 0.0900%, 0.0925%, 0.0950%, 0.0975%, 0.1000%, 0.1250%, 0.1500%, 0.1750%, 0.2000%, 0.2250%, 0.2500%, 0.2750%, 0.3000%, 0.3250%, 0.3500%, 0.3750%, 0.4000%, 0.4250%, 0.4500%, 0.4750%, 0.5000%, 0.5250%, 0.0550%, 0.5750%, 0.6000%, 0.6250%, 0.6500%, 0.6750%, 0.7000%, 0.7250%, 0.7500%, 0.7750%, 0.8000%, 0.8250%, 0.8500%, 0.8750%, 0.9000%, 0.9250%, 0.9500%, 0.9750%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5%, 4.6%, 4.7%, 4.8%, 4.9%, 5.0%, 5.1%, 5.2%, 5.3%, 5.4%, 5.5%, 5.6%, 5.7%, 5.8%, 5.9%, 6.0%, 6.1%, 6.2%, 6.3%, 6.4%, 6.5%, 6.6%, 6.7%, 6.8%, 6.9%, 7.0%, 7.1%, 7.2%, 7.3%, 7.4%, 7.5%, 7.6%, 7.7%, 7.8%, 7.9%, 8.0%, 8.1%, 8.2%, 8.3%, 8.4%, 8.5%, 8.6%, 8.7%, 8.8%, 8.9%, 9.0%, 9.1%, 9.2%, 9.3%, 9.4%, 9.5%, 9.6%, 9.7%, 9.8%, 9.9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50%, 60%, 65%, 70%, 75%, 80%, 85%, 90%, 95%, or 99% or any range derivable therein, of at least one of the ingredients that are mentioned throughout the specification and claims. In non-limiting aspects, the percentage can be calculated by weight or volume of the total composition. A person of ordinary skill in the art would understand that the concentrations can vary depending on the addition, substitution, and/or subtraction of ingredients in a given composition.

### F. Additional Ingredients

In addition to the combination of ingredients disclosed by the inventors, the first and second compositions can also include additional ingredients such as cosmetic ingredients and pharmaceutical active ingredients. Non-limiting examples of these additional ingredients are described in the following subsections.

### 1. Cosmetic Ingredients

The CTFA International Cosmetic Ingredient Dictionary and Handbook (2004 and 2008) describes a wide variety of non-limiting cosmetic ingredients that can be used in the context of the present invention. Examples of these ingredient classes include: fragrances (artificial and natural), adsorbents, lubricants, solvents, moisturizers (including, e.g., emollients, humectants, film formers, occlusive agents, and agents that affect the natural moisturization mechanisms of the skin), water-repellants, UV absorbers (physical and chemical absorbers such as paraaminobenzoic acid (“PABA”) and corresponding PABA derivatives, titanium dioxide, zinc oxide, etc.), essential oils, vitamins (e.g. A, B, C, D, E, and K), trace metals (e.g. zinc, calcium and selenium), anti-irritants (e.g. steroids and non-steroidal anti-inflammatories), botanical extracts (e.g. aloe vera, chamomile, cucumber extract, ginkgo biloba, ginseng, and rosemary), anti-microbial agents, antioxidants (e.g., BHT and tocopherol), chelating agents (e.g., disodium EDTA and tetrasodium EDTA), preservatives (e.g., methylparaben and propylparaben), pH adjusters (e.g., sodium hydroxide and citric acid), absorbents (e.g., aluminum starch octenylsuccinate, kaolin, corn starch, oat starch, cyclodextrin, talc, and zeolite), skin bleaching and lightening agents (e.g., hydroquinone and niacinamide lactate), humectants (e.g., sorbitol, urea, and manitol), exfoliants, waterproofing agents (e.g., magnesium/aluminum hydroxide stearate), skin conditioning agents (e.g., aloe extracts, allantoin, bisabolol, ceramides, dimethicone, hyaluronic acid, and dipotassium glycyrrhizate). Non-limiting examples of some of these ingredients are provided in the following subsections.

a. UV Absorption Agents

UV absorption agents that can be used in combination with the compositions of the present invention include chemical and physical sunblocks. Non-limiting examples of chemical sunblocks that can be used include para-aminobenzoic acid (PABA), PABA esters (glyceryl PABA, amyldimethyl PABA and octyldimethyl PABA), butyl PABA, ethyl PABA, ethyl dihydroxypropyl PABA, benzophenones (oxybenzone, sulisobenzone, benzophenone, and benzophenone-1 through 12), cinnamates (octyl methoxycinnamate, isoamyl p-methoxycinnamate, octylmethoxy cinnamate, cinoxate, diisopropyl methyl cinnamate, DEA-methoxycinnamate, ethyl diisopropylcinnamate, glyceryl octanoate dimethoxycinnamate and ethyl methoxycinnamate), cinnamate esters, salicylates (homomethyl salicylate, benzyl salicylate, glycol salicylate, isopropylbenzyl salicylate, etc.), anthranilates, ethyl urocanate, homosalate, octisalate, dibenzoylmethane derivatives (e.g., avobenzone), octocrylene, octyl triazone, digalloy trioleate, glyceryl aminobenzoate, lawsone with dihydroxyacetone, ethylhexyl triazone, dioctyl butamido triazone, benzylidene malonate polysiloxane, terephthalylidene dicamphor sulfonic acid, disodium phenyl dibenzimidazole tetrasulfonate, diethylamino hydroxybenzoyl hexyl benzoate, bis diethylamino hydroxybenzoyl benzoate, bis benzoxazoylphenyl ethylhexylimino triazine, drometrizole trisiloxane, methylene bis-benzotriazolyl tetramethylbutyiphenol, and bis-ethylhexyloxyphenol methoxyphenyltriazine, 4-methylbenzylidenecamphor, and isopentyl 4-methoxycinnamate. Non-limiting examples of physical sunblocks include, kaolin, talc, petrolatum and metal oxides (e.g., titanium dioxide and zinc oxide).

b. Moisturizing Agents

Non-limiting examples of moisturizing agents that can be used with the compositions of the present invention include amino acids, chondroitin sulfate, diglycerin, erythritol, fructose, glucose, glycerin, glycerol polymers, glycol, 1,2,6-hexanetriol, honey, hyaluronic acid, hydrogenated honey, hydrogenated starch hydrolysate, inositol, lactitol, maltitol, maltose, mannitol, natural moisturizing factor, PEG-15 butanediol, polyglyceryl sorbitol, salts of pyrollidone carboxylic acid, potassium PCA, propylene glycol, sodium glucuronate, sodium PCA, sorbitol, sucrose, trehalose, urea, and xylitol.

Other examples include acetylated lanolin, acetylated lanolin alcohol, alanine, algae extract, aloe barbadensis, aloe-barbadensis extract, aloe barbadensis gel, althea officinalis extract, apricot (prunus armeniaca) kernel oil, arginine, arginine aspartate, arnica montana extract, aspartic acid, avocado (persea gratissima) oil, barrier sphingolipids, butyl alcohol, beeswax, behenyl alcohol, beta-sitosterol, birch (betula alba) bark extract, borage (borago officinalis) extract, butcherbroom (ruscus aculeatus) extract, butylene glycol, calendula officinalis extract, calendula officinalis oil, candelilla (euphorbia cerifera) wax, canola oil, caprylic/capric triglyceride, cardamon (elettaria cardamomum) oil, carnauba (copernicia cerifera) wax, carrot (daucus carota sativa) oil, castor (ricinus communis) oil, ceramides, ceresin, ceteareth-5, ceteareth-12, ceteareth-20, cetearyl octanoate, ceteth-20, ceteth-24, cetyl acetate, cetyl octanoate, cetyl palmitate, chamomile (anthemis nobilis) oil, cholesterol, cholesterol esters, cholesteryl hydroxystearate, citric acid, clary (salvia sclarea) oil, cocoa (theobroma cacao) butter, coco-caprylate/caprate, coconut (cocos nucifera) oil, collagen, collagen amino acids, corn (zea mays)oil, fatty acids, decyl oleate, dimethicone copolyol, dimethiconol, dioctyl adipate, dioctyl succinate, dipentaerythrityl hexacaprylate/hexacaprate, DNA, erythritol, ethoxydiglycol, ethyl linoleate, eucalyptus globulus oil, evening primrose (oenothera biennis) oil, fatty acids, geranium maculatum oil, glucosamine, glucose glutamate, glutamic acid, glycereth-26, glycerin, glycerol, glyceryl distearate, glyceryl hydroxystearate, glyceryl laurate, glyceryl linoleate, glyceryl myristate, glyceryl oleate, glyceryl stearate, glyceryl stearate SE, glycine, glycol stearate, glycol stearate SE, glycosaminoglycans, grape (vitis vinifera) seed oil, hazel (corylus americana) nut oil, hazel (corylus avellana) nut oil, hexylene glycol, hyaluronic acid, hybrid safflower (carthamus tinctorius) oil, hydrogenated castor oil, hydrogenated coco-glycerides, hydrogenated coconut oil, hydrogenated lanolin, hydrogenated lecithin, hydrogenated palm glyceride, hydrogenated palm kernel oil, hydrogenated soybean oil, hydrogenated tallow glyceride, hydrogenated vegetable oil, hydrolyzed collagen, hydrolyzed elastin, hydrolyzed glycosaminoglycans, hydrolyzed keratin, hydrolyzed soy protein, hydroxylated lanolin, hydroxyproline, isocetyl stearate, isocetyl stearoyl stearate, isodecyl oleate, isopropyl isostearate, isopropyl lanolate, isopropyl myristate, isopropyl palmitate, isopropyl stearate, isostearamide DEA, isostearic acid, isostearyl lactate, isostearyl neopentanoate, jasmine (jasminum officinale) oil, jojoba (buxus chinensis) oil, kelp, kukui (aleurites moluccana) nut oil, lactamide MEA, laneth-16, laneth-10 acetate, lanolin, lanolin acid, lanolin alcohol, lanolin oil, lanolin wax, lavender (lavandula angustifolia) oil, lecithin, lemon (citrus medica limonum) oil, linoleic acid, linolenic acid, macadamia ternifolia nut oil, maltitol, matricaria (chamomilla recutita) oil, methyl glucose sesquistearate, methylsilanol PCA, mineral oil, mink oil, mortierella oil, myristyl lactate, myristyl myristate, myristyl propionate, neopentyl glycol dicaprylate/dicaprate, octyldodecanol, octyldodecyl myristate, octyldodecyl stearoyl stearate, octyl hydroxystearate, octyl palmitate, octyl salicylate, octyl stearate, oleic acid, olive (olea europaea) oil, orange (citrus aurantium dulcis) oil, palm (elaeis guineensis) oil, palmitic acid, pantethine, panthenol, panthenyl ethyl ether, paraffin, PCA, peach (prunus persica) kernel oil, peanut (arachis hypogaea) oil, PEG-8 C12-18 ester, PEG-15 cocamine, PEG-150 distearate, PEG-60 glyceryl isostearate, PEG-5 glyceryl stearate, PEG-30 glyceryl stearate, PEG-7 hydrogenated castor oil, PEG-40 hydrogenated castor oil, PEG-60 hydrogenated castor oil, PEG-20 methyl glucose sesquistearate, PEG40 sorbitan peroleate, PEG-5 soy sterol, PEG-10 soy sterol, PEG-2 stearate, PEG-8 stearate, PEG-20 stearate, PEG-32 stearate, PEG40 stearate, PEG-50 stearate, PEG-100 stearate, PEG-150 stearate, pentadecalactone, peppermint (mentha piperita) oil, petrolatum, phospholipids, polyamino sugar condensate, polyglyceryl-3 diisostearate, polyquaternium-24, polysorbate 20, polysorbate 40, polysorbate 60, polysorbate 80, polysorbate 85, potassium myristate, potassium palmitate, propylene glycol, propylene glycol dicaprylate/dicaprate, propylene glycol dioctanoate, propylene glycol dipelargonate, propylene glycol laurate, propylene glycol stearate, propylene glycol stearate SE, PVP, pyridoxine dipalmitate, retinol, retinyl palmitate, rice (oryza sativa) bran oil, RNA, rosemary (rosmarinus officinalis) oil, rose oil, safflower (carthamus tinctorius) oil, sage (salvia officinalis) oil, sandalwood (santalum album) oil, serine, serum protein, sesame (sesamum indicum) oil, shea butter (butyrospermum parkii), silk powder, sodium chondroitin sulfate, sodium hyaluronate, sodium lactate, sodium palmitate, sodium PCA, sodium polyglutamate, soluble collagen, sorbitan laurate, sorbitan oleate, sorbitan palmitate, sorbitan sesquioleate, sorbitan stearate, sorbitol, soybean (glycine soja) oil, sphingolipids, squalane, squalene, stearamide MEA-stearate, stearic acid, stearoxy dimethicone, stearoxytrimethylsilane, stearyl alcohol, stearyl glycyrrhetinate, stearyl heptanoate, stearyl stearate, sunflower (helianthus annuus) seed oil, sweet almond (prunus amygdalus dulcis) oil, synthetic beeswax, tocopherol, tocopheryl acetate, tocopheryl linoleate, tribehenin, tridecyl neopentanoate, tridecyl stearate, triethanolamine, tristearin, urea, vegetable oil, water, waxes, wheat (triticum vulgare) germ oil, and ylang ylang (cananga odorata) oil.

c. Antioxidants

Non-limiting examples of antioxidants that can be used with the compositions of the present invention include acetyl cysteine, ascorbic acid polypeptide, ascorbyl dipalmitate, ascorbyl methylsilanol pectinate, ascorbyl palmitate, ascorbyl stearate, BHA, BHT, t-butyl hydroquinone, cysteine, cysteine HCI, diamylhydroquinone, di-t-butylhydroquinone, dicetyl thiodipropionate, dioleyl tocopheryl methylsilanol, disodium ascorbyl sulfate, distearyl thiodipropionate, ditridecyl thiodipropionate, dodecyl gallate, erythorbic acid, esters of ascorbic acid, ethyl ferulate, ferulic acid, gallic acid esters, hydroquinone, isooctyl thioglycolate, kojic acid, magnesium ascorbate, magnesium ascorbyl phosphate, methylsilanol ascorbate, natural botanical anti-oxidants such as green tea or grape seed extracts, nordihydroguaiaretic acid, octyl gallate, phenylthioglycolic acid, potassium ascorbyl tocopheryl phosphate, potassium sulfite, propyl gallate, quinones, rosmarinic acid, sodium ascorbate, sodium bisulfite, sodium erythorbate, sodium metabisulfite, sodium sulfite, superoxide dismutase, sodium thioglycolate, sorbityl furfural, thiodiglycol, thiodiglycolamide, thiodiglycolic acid, thioglycolic acid, thiolactic acid, thiosalicylic acid, tocophereth-5, tocophereth-10, tocophereth-12, tocophereth-18, tocophereth-50, tocopherol, tocophersolan, tocopheryl acetate, tocopheryl linoleate, tocopheryl nicotinate, tocopheryl succinate, and tris(nonylphenyl)phosphite.

d. Structuring Agents

In other non-limiting aspects, the compositions of the present invention can include a structuring agent. Structuring agent, in certain aspects, assist in providing rheological characteristics to the composition to contribute to the composition's stability. In other aspects, structuring agents can also function as an emulsifier or surfactant. Non-limiting examples of structuring agents include stearic acid, palmitic acid, stearyl alcohol, cetyl alcohol, behenyl alcohol, stearic acid, palmitic acid, the polyethylene glycol ether of stearyl alcohol having an average of about 1 to about 21 ethylene oxide units, the polyethylene glycol ether of cetyl alcohol having an average of about 1 to about 5 ethylene oxide units, and mixtures thereof.

e. Emulsifiers

In certain aspects of the present invention, the compositions do not include an emulsifier. In other aspects, however, the compositions can include one or more emulsifiers. Emulsifiers can reduce the interfacial tension between phases and improve the formulation and stability of an emulsion. The emulsifiers can be nonionic, cationic, anionic, and zwitterionic emulsifiers (See McCutcheon's (1986); U.S. Pat. Nos. 5,011,681; 4,421,769; 3,755,560). Non-limiting examples include esters of glycerin, esters of propylene glycol, fatty acid esters of polyethylene glycol, fatty acid esters of polypropylene glycol, esters of sorbitol, esters of sorbitan anhydrides, carboxylic acid copolymers, esters and ethers of glucose, ethoxylated ethers, ethoxylated alcohols, alkyl phosphates, polyoxyethylene fatty ether phosphates, fatty acid amides, acyl lactylates, soaps, TEA stearate, DEA oleth-3 phosphate, polyethylene glycol 20 sorbitan monolaurate (polysorbate 20), polyethylene glycol 5 soya sterol, steareth-2, steareth-20, steareth-21, ceteareth-20, PPG-2 methyl glucose ether distearate, ceteth-10, polysorbate 80, cetyl phosphate, potassium cetyl phosphate, diethanolamine cetyl phosphate, polysorbate 60, glyceryl stearate, PEG-100 stearate, and mixtures thereof.

f. Silicone Containing Compounds

In non-limiting aspects, silicone containing compounds include any member of a family of polymeric products whose molecular backbone is made up of alternating silicon and oxygen atoms with side groups attached to the silicon atoms. By varying the —Si—O— chain lengths, side groups, and crosslinking, silicones can be synthesized into a wide variety of materials. They can vary in consistency from liquid to gel to solids.

The silicone containing compounds that can be used in the context of the present invention include those described in this specification or those known to a person of ordinary skill in the art. Non-limiting examples include silicone oils (e.g., volatile and non-volatile oils), gels, and solids. In certain aspects, the silicon containing compounds includes a silicone oils such as a polyorganosiloxane. Non-limiting examples of polyorganosiloxanes include dimethicone, cyclomethicone, polysilicone-11, phenyl trimethicone, trimethylsilylamodimethicone, stearoxytrimethylsilane, or mixtures of these and other organosiloxane materials in any given ratio in order to achieve the desired consistency and application characteristics depending upon the intended application (e.g., to a particular area such as the skin, hair, or eyes). A “volatile silicone oil” includes a silicone oil have a low heat of vaporization, i.e. normally less than about 50 cal per gram of silicone oil. Non-limiting examples of volatile silicone oils include: cyclomethicones such as Dow Corning 344 Fluid, Dow Corning 345 Fluid, Dow Corning 244 Fluid, and Dow Corning 245 Fluid, Volatile Silicon 7207 (Union Carbide Corp., Danbury, Conn.); low viscosity dimethicones, i.e. dimethicones having a viscosity of about 50 cst or less (e.g., dimethicones such as Dow Corning 200-0.5 cst Fluid). The Dow Corning Fluids are available from Dow Corning Corporation, Midland, Michigan. Cyclomethicone and dimethicone are described in the Third Edition of the CTFA Cosmetic Ingredient Dictionary (incorporated by reference) as cyclic dimethyl polysiloxane compounds and a mixture of fully methylated linear siloxane polymers end-blocked with trimethylsiloxy units, respectively. Other non-limiting volatile silicone oils that can be used in the context of the present invention include those available from General Electric Co., Silicone Products Div., Waterford, N.Y. and SWS Silicones Div. of Stauffer Chemical Co., Adrian, Mich.

g. Essential Oils

Essential oils include oils derived from herbs, flowers, trees, and other plants. Such oils are typically present as tiny droplets between the plant's cells, and can be extracted by several method known to those of skill in the art (e.g., steam distilled, enfleurage (i.e., extraction by using fat), maceration, solvent extraction, or mechanical pressing). When these types of oils are exposed to air they tend to evaporate (i.e., a volatile oil). As a result, many essential oils are colorless, but with age they can oxidize and become darker. Essential oils are insoluble in water and are soluble in alcohol, ether, fixed oils (vegetal), and other organic solvents. Typical physical characteristics found in essential oils include boiling points that vary from about 160° to 240° C. and densities ranging from about 0.759 to about 1.096.

Essential oils typically are named by the plant from which the oil is found. For example, rose oil or peppermint oil are derived from rose or peppermint plants, respectively. Non-limiting examples of essential oils that can be used in the context of the present invention include sesame oil, macadamia nut oil, tea tree oil, evening primrose oil, Spanish sage oil, Spanish rosemary oil, coriander oil, thyme oil, pimento berries oil, rose oil, anise oil, balsam oil, bergamot oil, rosewood oil, cedar oil, chamomile oil, sage oil, clary sage oil, clove oil, cypress oil, eucalyptus oil, fennel oil, sea fennel oil, frankincense oil, geranium oil, ginger oil, grapefruit oil, jasmine oil, juniper oil, lavender oil, lemon oil, lemongrass oil, lime oil, mandarin oil, marjoram oil, myrrh oil, neroli oil, orange oil, patchouli oil, pepper oil, black pepper oil, petitgrain oil, pine oil, rose otto oil, rosemary oil, sandalwood oil, spearmint oil, spikenard oil, vetiver oil, wintergreen oil, or ylang ylang. Other essential oils known to those of skill in the art are also contemplated as being useful within the context of the present invention.

h. Thickening Agents

Thickening agents, including thickener or gelling agents, include substances which that can increase the viscosity of a composition. Thickeners includes those that can increase the viscosity of a composition without substantially modifying the efficacy of the active ingredient within the composition. Thickeners can also increase the stability of the compositions of the present invention. In certain aspects of the present invention, thickeners include hydrogenated polyisobutene or trihydroxystearin, or a mixture of both.

Non-limiting examples of additional thickening agents that can be used in the context of the present invention include carboxylic acid polymers, crosslinked polyacrylate polymers, polyacrylamide polymers, polysaccharides, and gums. Examples of carboxylic acid polymers include crosslinked compounds containing one or more monomers derived from acrylic acid, substituted acrylic acids, and salts and esters of these acrylic acids and the substituted acrylic acids, wherein the crosslinking agent contains two or more carbon-carbon double bonds and is derived from a polyhydric alcohol (see U.S. Pat. Nos. 5,087,445; 4,509,949; 2,798,053; CTFA International Cosmetic Ingredient Dictionary, Fourth edition, 1991, pp. 12 and 80). Examples of commercially available carboxylic acid polymers include carbomers, which are homopolymers of acrylic acid crosslinked with allyl ethers of sucrose or pentaerytritol (e.g., Carbopol™ 900 series from B. F. Goodrich).

Non-limiting examples of crosslinked polyacrylate polymers include cationic and nonionic polymers. Examples are described in U.S. Pat. Nos. 5,100,660 ; 4,849,484; 4,835,206; 4,628,078; 4,599,379).

Non-limiting examples of polyacrylamide polymers (including nonionic polyacrylamide polymers including substituted branched or unbranched polymers) include polyacrylamide, isoparaffin and laureth-7, multi-block copolymers of acrylamides and substituted acrylamides with acrylic acids and substituted acrylic acids.

Non-limiting examples of polysaccharides include cellulose, carboxymethyl hydroxyethylcellulose, cellulose acetate propionate carboxylate, hydroxyethylcellulose, hydroxyethyl ethylcellulose, hydroxypropylcellulose, hydroxypropyl methylcellulose, methyl hydroxyethylcellulose, microcrystalline cellulose, sodium cellulose sulfate, and mixtures thereof. Another example is an alkyl substituted cellulose where the hydroxy groups of the cellulose polymer is hydroxyalkylated (preferably hydroxy ethylated or hydroxypropylated) to form a hydroxyalkylated cellulose which is then further modified with a C₁₀-C₃₀ straight chain or branched chain alkyl group through an ether linkage. Typically these polymers are ethers of C₁₀-C₃₀ straight or branched chain alcohols with hydroxyalkylcelluloses. Other useful polysaccharides include scleroglucans comprising a linear chain of (1-3) linked glucose units with a (1-6) linked glucose every three unit.

Non-limiting examples of gums that can be used with the present invention include acacia, agar, algin, alginic acid, ammonium alginate, amylopectin, calcium alginate, calcium carrageenan, carnitine, carrageenan, dextrin, gelatin, gellan gum, guar gum, guar hydroxypropyltrimonium chloride, hectorite, hyaluroinic acid, hydrated silica, hydroxypropyl chitosan, hydroxypropyl guar, karaya gum, kelp, locust bean gum, natto gum, potassium alginate, potassium carrageenan, propylene glycol alginate, sclerotium gum, sodium carboyxmethyl dextran, sodium carrageenan, tragacanth gum, xanthan gum, and mixtures thereof.

i. Preservatives

Non-limiting examples of preservatives that can be used in the context of the present invention include quaternary ammonium preservatives such as polyquaternium-1 and benzalkonium halides (e.g., benzalkonium chloride (“BAC”) and benzalkonium bromide), parabens (e.g., methylparabens and propylparabens), phenoxyethanol, benzyl alcohol, chlorobutanol, phenol, sorbic acid, thimerosal or combinations thereof.

### 2. Pharmaceutical Ingredients

Pharmaceutical active agents are also contemplated as being useful with the compositions of the present invention. Non-limiting examples of pharmaceutical active agents include anti-acne agents, agents used to treat rosacea, analgesics, anesthetics, anorectals, antihistamines, anti-inflammatory agents including non-steroidal anti-inflammatory drugs, antibiotics, antifungals, antivirals, antimicrobials, anti-cancer actives, scabicides, pediculicides, antineoplastics, antiperspirants, antipruritics, antipsoriatic agents, antiseborrheic agents, biologically active proteins and peptides, burn treatment agents, cauterizing agents, depigmenting agents, depilatories, diaper rash treatment agents, enzymes, hair growth stimulants, hair growth retardants including DFMO and its salts and analogs, hemostatics, kerotolytics, canker sore treatment agents, cold sore treatment agents, dental and periodontal treatment agents, photosensitizing actives, skin protectant/barrier agents, steroids including hormones and corticosteroids, sunburn treatment agents, sunscreens, transdermal actives, nasal actives, vaginal actives, wart treatment agents, wound treatment agents, wound healing agents, etc.

### EXAMPLES

The following examples are included to demonstrate certain non-limiting aspects of the invention. It should be appreciated by those of skill in the art that the techniques disclosed in the examples which follow represent techniques discovered by the inventor to function well in the practice of the invention. However, those of skill in the art should, in light of the present disclosure, appreciate that many changes can be made in the specific embodiments which are disclosed and still obtain a like or similar result without departing from the spirit and scope of the invention.

### Example 1

The Table 1 formulation is formulated as a BB cream, in which the continuous phase is water and the discontinuous phase comprises titanium dioxide evenly dispersed through the continuous phase. The formulation is white in color.

TABLE 1*

| Phase | Ingredient | % w/w |
|---|---|---|
| A | Dionized Water | q.s. |
| | Titanium Dioxide (water dispersible) | 10 |
| | Glycerin | 2 |
| | Pentylene Glycol | 4 |
| B | Aristoflex AVC** | 0.5 |
| C | Capric/Caprylic Triglycerides/Magnolia Flower | 2 |
| | Extract/Bisabalol*** | |
| | Octomethylcinnamate (OMC) | 5 |
| | Hydresia SF2 | 10 |
| D | Optiphen Plus | 1.3 |
| | TOTAL | 100 |

*Formulation prepared by following procedure: (1) added phase A ingredients (in order as listed) into a main vessel and mixed under propeller: (2) added phase B ingredient to main vessel slowly, allowing phase B ingredient to disperse and mixture to thicken; (3) using a second vessel, added capric/caprylic triglycerides/magnolia flower extract/bisabalol mixture with OMC and then added Hydresia SF2 slowly under propeller; (4) added second vessel contents to main vessel contents and mixed under propeller; (5) added phase D ingredient under propeller until homogenous white cream is produced.
**Aristoflex AVC is a mixture of Ammonium Acryloyldimethyltaurate/VP Copolymer, which can be purchased from Aako (BV) (The Netherlands).
***Mixture of Capric/Caprylic Triglycerides/Magnolia Flower Extract/Bisabalol included 85% w/w Capric/Caprylic Triglycerides, 5% w/w Magnolia Flower Extract, and 10% w/w Bisabalol.

### Example 2

Water-dissolvable hydroxypropyl methyl cellulose (HPMC) films having a thickness of two millimeters and three millimeters and also having a beige color were added to the Table 1 formulation. The three millimeter films took longer to dissolve when compared to the two millimeter films in the Table 1 formulation. Both of the two and three millimeter films dissolved within the formulation and evenly dispersed their respective colorant contents. The formulations changed color from white to beige without the appearance of any coloring streaks. The HPMC water-dissolvable films used in this study were provided by the Dow Chemical Company. Processes for making HPMC water-dissolvable films are described in “Film-Forming Polymers in Fast-Dissolve Oral Films,” by Chen et al., a Dow Chemical Company publication (2006) (accessible at http://msdssearch.dow.com/PublishedLiteratureDOWCOM/dh_{—}0062/0901b803800629f0.pdf?fil epath=dowexcipients/pdfs/noreg/198-02161.pdf&fromPage=GetDoc) and “Edible Films Applications,” Methocel Food Gums (accessible at http://msdssearch.dow.com/PublishedLiteratureDOWCOM/dh_{—}0032/0901b80380032eff.pdf?file path=/194-01283.pdf&fromPage=GetDoc), both of which are incorporated by reference. Additional water-dissolvable films having various colors are commercially available from suppliers such as Aicello (Japan) under the trade name Solublon® Water Soluble Film and MonoSol (USA) under the trade names MonoDose™.

### Example 3

The following is a non-limiting process that one can use to prepare a water-dissolvable film or strip of the present invention. The following Table 2 describes a hydroxypropylcellulose (HPC) derived water-dissolvable film mixture (note that other cellulose derivatives can be used in place of HPC).

TABLE 2

| Ingredient | % w/w |
|---|---|
| Polyvinyl alcohol (Celvol 540 PVA) | 2.0 |
| Glucose | 1.0 |
| HPC (Klucel EF) | 3.1 |
| Water | 81.3 |
| Carragheenan | 1.0 |
| Colorant mixture* | 3 |
| Esterified corn starch (Dry Flo Plus) | 1.6 |
| Glycerol | 3.1 |
| Polyethylene glycol (PEG) 400 | 1.9 |
| PEG-12 Dimethicone (Silsoft 880) | 2.0 |
| TOTAL | 100 |

*The colorant mixture can be a single colorant or a mixture of colorants to impart a particular color or shade of color to the film. The beige color of the films produced in Example 2 were from a dry mixture of the following: 87.9% w/w titanium dioxide; 7.8% w/w yellow; 1.9% w/w red; 1.5% w/w black; and 0.9% w/w ultra blue.

A film or strip of the present invention using the above formula can be prepared as follows. First, the cellulose polymer and the polyvinyl alcohol are mixed in the water to create a dispersion. The mixing process can include stirring at a speed and for a period of time that are necessary to obtain a homogeneous gel. Stirring (1200 to 1600 rpm) using a turbine mixer (Rayneri) can be used. Second, the PEG-12 dimethicone, the colorant mixture, and the carragheenan can be added under continued mixing until a homogenized mixture is obtained. Third, the esterified corn starch, the PEG 400, the glucose, and the glycerol can be added under continuous stirring until homogenous mixture obtained. The stirring procedure can be done at room temperature (20-25° C.). Fourth, the resulting colored mixture can be layered onto the nonsiliconized face of a sheet of Silphan S100 M44A, using a film drawer. The apparatus used for this step can be an automatic film-applying apparatus from Braive Instruments. The wet thickness chosen can vary as desired to create a particular thickness of the resulting film or strip (e.g., under this process around 500 μm can be used). This can be followed by drying the wet mixture in a tunnel with circulation of hot air at a temperature of approximately 50° C., which results in the produced film. Said film can then be cut into a desired shape (e.g., strips, squares, circular patents, rectangles, etc.).

1. A kit for preparing a colored topical skin composition, the kit comprising:
(a) a first composition comprising an aqueous continuous phase comprising at least 50% by weight of water and a discontinuous phase comprising titanium dioxide or zinc oxide or a combination thereof in an amount sufficient to impart a white color appearance to the first composition, wherein the titanium dioxide or zinc oxide is suspended within the aqueous continuous phase, and wherein the color of the first composition is white; and
(b) a second composition in the form of a water-dissolvable film, wherein the film has a color other than white,
wherein the second composition is capable of dissolving within the first composition and imparting a color other than white to the first composition when the first and second compositions are mixed together.

2. The kit of claim 1, wherein the first composition comprises 60% to 75% w/w of water.

3. The kit of claim 2, wherein the first composition comprises 8 to 12% w/w of titanium dioxide or zinc oxide or a combination thereof.

4. The kit of claim 3, wherein the first composition further comprises 0.5 to 2% w/w of a gelling agent.

5. The kit of claim 4, wherein the first composition further comprises 5 to 15% w/w of a moisturizer.

6. The kit of claim 1, wherein the viscosity of the first composition is 100 cps to 5,000 cps, as measured on a Brookfield Viscometer using a TC spindle at 2.5 rpm at 25° C.

7. The kit of claim 1, wherein the water-dissolvable film is a cellulose-based film.

8. The kit of claim 8, wherein the cellulose-based film is a hydroxypropyl cellulose (HPC) based film, a hydroxypropylmethyl cellulose (HPMC) based film, or a carboyxymethyl cellulose (CMC) based film.

9. The kit of claim 8, wherein the water-dissolvable film comprises a blend of pigments that are capable of imparting a color other than white to the cellulose-based film.

10. The kit of claim 9, wherein the color of the water-dissolvable film is red, green, blue, yellow, orange, brown, or black.

11. The kit of claim 1, wherein the water-dissolvable film has a thickness ranging from 1 to 3 millimeters.

12. The kit of claims 1, wherein the first composition is comprised in a first container and the second composition is comprised in a second container.

13. The kit of claim 12, wherein the first and second containers are comprised within a third container.

14. The kit of claim 1, comprising at least a third composition in the form of a water-dissolvable film, wherein the third composition has a different color than the second composition.

15. The kit of claim 1, comprising at least a third composition in the form of a water-dissolvable film, wherein the third composition has the same color as the second composition.

16. A method of preparing a composition for topical application to skin with the kit of claim 1, the method comprising mixing the first composition and the second composition together, wherein the second composition dissolves within the first composition, and wherein the color of the first composition changes from white to a color other than white.

17. The method of claim 16, wherein the color of the first composition, after mixing with the second composition, is substantially identical to the color of a user's skin color.

18. The method of claim 16, wherein the first composition, after mixing with the second composition, is blue, green, red, yellow, orange, or brown.

19. The method of claim 16, comprising at least a third composition in the form of a water-dissolvable film, wherein the third composition has a different color than the second composition.

20. The method of claim 16, comprising at least a third composition in the form of a water-dissolvable film, wherein the third composition has the same color as the second composition.
